(19) United States
(12) Patent Application Publication
(10) Pub. No.: US 2025/0095851 A1
(43) Pub. Date: Mar. 20, 2025

(54) A SYSTEM FOR DETECTION AND CLASSIFICATION OF CARDIAC DISEASES USING CUSTOM DEEP NEURAL NETWORK TECHNIQUES

(71) Applicant: Vijendra VENKATESH, Kolar (IN)

(72) Inventors: Vijendra VENKATESH, Kolar (IN); Meghana KULKARNI, Kolar (IN)

(21) Appl. No.: 18/727,718

(22) PCT Filed: Jan. 19, 2023
(86) PCT No.: PCT/IB2023/050447

§ 371 Date: Jul. 10, 2024

(30) Foreign Application Priority Data
Jan. 12, 2022 (IN) 202241001730

(51) Int. Cl. G16H 50/20; G16H 30/40

(52) U.S. Cl. CPC G16H 50/20; G16H 30/40

(57) ABSTRACT

The invention discloses a system for detection and classification of cardiac diseases using deep neural network techniques, wherein the system (100) comprises filtration module (101) for filtering one-dimensional Electrocardiogram (ECG) data. The filtered ECG data is provided to feature extraction module (102) for extracting a set of pre-defined features from the PQRST complex, wherein the extracted features are classified by the classification module (103) for the purpose of cardiac disease detection using interval and peak detection techniques. Further, the classified one-dimensional ECG data is converted into a two-dimensional spectrogram image by the conversion module (104). The two-dimensional spectrogram image is passed through custom deep neural networks and the diagnostic results may be accessed through a remote server module (105) and viewed by the individuals on a user interface device.

### DESCRIPTION OF THE INVENTION

### Technical Field of the Invention

The present invention discloses a system for detection and classification of cardiac diseases using custom deep neural network techniques. The invention particularly relates to a mechanism of converting classified one-dimensional cardiac data into two-dimensional spectrogram images for facilitating the classification of a large number of cardiac diseases thereby improving the efficiency and robustness of the system.

### Background of the Invention

Early detection of cardiac related illness and subsequent treatment is one of the most crucial tasks which is capable of reducing the fatality rate especially in developing countries such as India which largely comprises of people from lower economic sectors who do not have affordability and accessibility to healthcare facilities. The existing state of the art for monitoring cardiac health is limited to identifying very few irregularities and fails to detect more crucial conditions such as Hyperkalemia, Hypercalcemia, Hypokalemia, Hypocalcemia and so on. Presently, there are no one-stop, accessible and cost-effective solution for the detection of a large number of cardiac diseases.

The U.S. Pat. No. 9,339,241B2 titled “Assessment and prediction of cardiovascular status during cardiac arrest and the post-resuscitation period using signal processing and machine learning” relates to real-time, short-term analysis of ECG, by using multiple signal processing and machine learning techniques, is used to determine counter shock success in defibrillation. Combinations of measures when used with machine learning algorithms readily predict successful resuscitation, guide therapy and predict complications. In terms of guiding resuscitation, they may serve as indicators and when to provide counter shocks and at what energy levels they should be provided as well as to serve as indicators of when certain drugs should be provided (in addition to their doses). For cardiac arrest, the system is meant to run in real time during all current resuscitation procedures including post-resuscitation care to detect deterioration for guiding care such as therapeutic hypothermia.

The U.S. Pat. No. 10,542,889B2 titled “Systems, methods, and devices for remote health monitoring and management” relates to a remote health monitoring system, method and device. The systems utilize one or more sensors, data aggregation and transmission units, mobile computing devices, processing, analytics and storage (PAS) units, and a framework based on a novel location- and power-aware communication systems and analytics to notify and manage patient health. Methods to transmit data to a PAS unit through the patients' smart phone that is connected to internet, abnormality detection in the data, advanced analytical diagnostics and communication system between the health service provider (HSP) and patient are also provided. The health monitoring systems, methods and devices allows for continuous monitoring of the patient without disrupting their normal lives, provides access even in sparsely connected and remote regions which lack good healthcare facilities, allows intervention by specialized practitioners, and sharing of resource or information in the existing healthcare facilities.

Hence, there exists a need for a solution to classify and detect a larger number of cardiac disease than capable by the existing state of the art mechanisms.

### SUMMARY OF THE INVENTION

The present invention overcomes the drawbacks of the prior art by disclosing a system for detection and classification of cardiac diseases using deep neural network techniques, wherein the system comprises a filtration module for filtering one-dimensional Electrocardiogram (ECG) data. Further, the filtered ECG data is provided to a feature extraction module for extracting a set of pre-defined features from the PQRST complex, wherein the extracted features are classified by the classification module for the purpose of cardiac disease detection using interval and peak detection techniques. Further, the classified one-dimensional ECG data is converted into a two-dimensional spectrogram image by the conversion module. The two-dimensional spectrogram image may be accessed through a remote server module and viewed by one or more individuals on a user interface device.

The present invention provides a solution to the persistent issue of detecting very few number of cardiac irregularities or diseases using the existing state of the art. Presently, common irregularities such as arrhythmias, atrial fibrillation, cardiomyopathy are detected using the existing state of the art, however, the classification module in the system is capable of detecting complex cardiac diseases including dysrhythmia, supraventricular dysrhythmia, Sinus bradycardia, Sinus arrest, Sinus tachycardia, Atrial fibrillation, Atrioventricular Junction rhythm, AV conduction block, First degree heart block, second degree heart block, and third-degree cardiac arrest, Hyperkalemia, Hypercalcemia, Hypokalemia, Hypocalcemia diseases and so on. Additionally, the conversion of one-dimensional classified ECG data into two-dimensional spectrogram image by the conversion module enables the classification of a large number of cardiac diseases thereby improving the efficiency and robustness of the system.

### BRIEF DESCRIPTION OF THE DRAWINGS

The foregoing and other features of embodiments will become more apparent from the following detailed description of embodiments when read in conjunction with the accompanying drawings. In the drawings, like reference numerals refer to like elements.

FIG. 1 illustrates a block diagram of a system for detection and classification of cardiac diseases using deep neural network techniques.

### DETAILED DESCRIPTION OF THE INVENTION

Reference will now be made in detail to the description of the present subject matter, one or more examples of which are shown in FIGURES. Each example is provided to explain the subject matter and not a limitation. Various changes and modifications obvious to one skilled in the art to which the invention pertains are deemed to be within the spirit, scope and contemplation of the invention.

The present invention discloses a system for detection and classification of cardiac diseases using deep neural network techniques, wherein the system comprises a filtration module for filtering one-dimensional Electrocardiogram (ECG) data. Further, the filtered ECG data is provided to a feature extraction module for extracting a set of pre-defined features from the PQRST complex, wherein the extracted features are classified by the classification module for the purpose of cardiac disease detection using interval and peak detection techniques. Further, the classified one-dimensional ECG data is converted into a two-dimensional spectrogram image by the conversion module. The two-dimensional spectrogram image may be accessed through a remote server module and viewed by one or more individuals on a user interface device

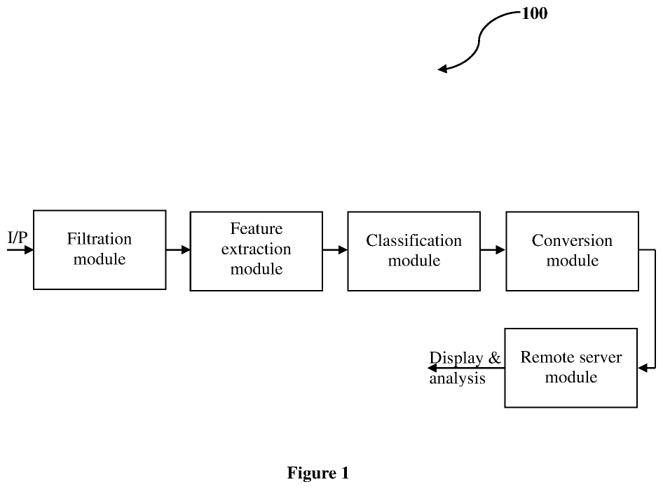

FIG. 1 illustrates a block diagram of a system for detection and classification of cardiac diseases using deep neural network techniques. The system (100) comprises a filtration module (101) for filtering one or more datasets pertaining to the one-dimensional Electrocardiogram (ECG) data obtained from a plurality of individuals, wherein the datasets may be derived from an extensive database comprising cardiac data pertaining to a plurality of individuals and the cardiac irregularities/diseases associated with them. The filtration module (101) filters the one-dimensional ECG datasets to eliminate the presence of noise and artifacts using a plurality of filtration techniques such as and not limited to Discrete Wavelet Transformation (DWT) techniques.

Further, the filtered one-dimensional ECG data is provided to a feature extraction module (102) for extracting a set of pre-defined features from the filtered one-dimensional ECG data using techniques such as fiducial points, adaptive thresholding, lifting based schemes and so on, wherein the extracted features include various combinations of the PQRST complex such as RR, SS, QRS complex, QT, ST segment values present in the one-dimensional ECG data.

Multiple combinations of the PQRST complex is extracted based on the disease to be identified. The extracted features from the one-dimensional ECG data is provided to a classification module (103) for the purpose of classifying the extracted features.

The classification module (103) employs interval and peak detection classification techniques for the purpose of data classification. Further, the classification module (103) compares the extracted features with one or more test data to match the Euclidean Distance, wherein if the Euclidean Distance of the extracted features and test data is equal, then no cardiac disease is detected. In an event where the Euclidean Distance of the extracted features and test data is unequal, then the presence of cardiac disease is detected. The classified data from the classification module (103) is provided to a conversion module (104) for converting classified features from one-dimensional ECG signal present in serial format into a matrix format representing a two-dimensional spectrogram image, wherein the two-dimensional spectrogram image is deployed into a custom layer of a neural network such as a hybrid model of a Convolutional Neural Network (CNN) and Recurrent Neural Network (RNN). Further, the two-dimensional spectrogram image is uploaded to a remote server module (105) using a wired or wireless network infrastructure for enabling the display and/or further analysis of the detected cardiac disease on one or more user interface devices such as mobile phone, tablet and so on. In one embodiment, the user interface device may be an independent and standalone device with a display unit developed for implementing the system (100).

The present invention provides a solution to the persistent issue of detecting very few number of cardiac irregularities or diseases using the existing state of the art. Presently, common irregularities such as arrhythmias, atrial fibrillation, cardiomyopathy are detected using the existing state of the art, however, the classification module (103) in the system (100) is capable of detecting complex cardiac diseases including dysrhythmia, supraventricular dysrhythmia, Sinus bradycardia, Sinus arrest, Sinus tachycardia, Atrial fibrillation, Atrioventricular Junction rhythm, AV conduction block, First degree heart block, second degree heart block, and third-degree cardiac arrest, Hyperkalemia, Hypercalcemia, Hypokalemia, Hypocalcemia diseases and so on. Additionally, the conversion of one-dimensional classified ECG data into two-dimensional spectrogram image by the conversion module (104) enables the classification of a large number of cardiac diseases thereby improving the efficiency and robustness of the system (100).

While at least one exemplary embodiment has been presented in the foregoing detailed description, it should be appreciated that a vast number of variations exist.

Reference numbers:

| Components | Reference Numbers |
|---|---|
| System | 100 |
| Filtration module | 101 |
| Feature extraction module | 102 |
| Classification module | 103 |
| Conversion module | 104 |
| Remote server module | 105 |

We claim:

1. A system for detection and classification of cardiac diseases using deep neural network techniques, the system (100) comprising:
a. a filtration module (101) for filtering one or more datasets pertaining to the one-dimensional Electrocardiogram (ECG) data obtained from a plurality of individuals, wherein the filtration module (101) filters the one-dimensional ECG datasets to eliminate the presence of noise and artifacts;
b. a feature extraction module (102) for extracting a set of pre-defined features from the filtered one-dimensional ECG data, wherein the extracted features include various combinations of the PQRST complex present in the one-dimensional ECG data;
c. a classification module (103) for classifying the extracted features from the filtered one-dimensional ECG data for the purpose of cardiac disease detection using interval and peak detection techniques, wherein the classification module (103):
i. compares the extracted features with one or more test data to match the Euclidean Distance, wherein if the Euclidean Distance of the extracted features and test data is:
1. equal, then no cardiac disease is detected;
2. unequal, then the presence of cardiac disease is detected;
d. a conversion module (104) for converting classified features from the one-dimensional ECG signal into a two-dimensional spectrogram image, wherein the conversion module (104) deploys the two-dimensional spectrogram image into a custom deep neural network;
e. a remote server module (105) for enabling the display of the detected cardiac disease on one or more user interface devices, wherein the remote server module (105) communicates with the conversion module (104) using a wired or wireless network infrastructure.

2. The system (100) as claimed in claim 1, wherein the one-dimensional ECG data is filtered by the filtration module (101) using Discrete Wavelet Transformation (DWT) techniques.

3. The system (100) as claimed in claim 1, wherein a plurality of combinations of the PQRST complex is extracted based on the disease to be identified.

4. The system (100) as claimed in claim 1, wherein the resultant two-dimensional spectrogram image from the conversion module (104) enables the classification of a large number of cardiac diseases thereby improving the efficiency and robustness of the system (100).
